(19) United States
(12) Patent Application Publication
(10) Pub. No.: US 2016/0220484 A1
(43) Pub. Date: Aug. 4, 2016

(54) APHRODISIAC ESSENTIAL OIL COMPOSITION AND ITS APPLICATION METHOD

(71) Applicant: Tien-Te Lee, Taipei (TW)

(72) Inventor: Tien-Te Lee, Taipei (TW)

(21) Appl. No.: 14/611,441

(22) Filed: Feb. 2, 2015

(51) Int. Cl. A61K 9/00; A61K 36/906; A61K 36/61; A61K 36/87; A61K 36/738; A61K 36/54; A61K 36/537; A61K 36/53; A61K 36/752; A61K 36/534; A61K 36/736

(52) U.S. Cl. CPC A61K 9/0034; A61K 36/534; A61K 36/906; A61K 36/61; A61K 36/87; A61K 36/736; A61K 36/54; A61K 36/537; A61K 36/53; A61K 36/752; A61K 36/738

(57) ABSTRACT

An aphrodisiac essential oil composition for directly applying to the body, or for use in a condom or paper towel to achieve the effect of helping increasing sexual desire is prepared by mixing 10˜90 parts of peppermint oil, 10˜90 parts of ginger oil, 10˜90 parts of tea tree oil, 10˜90 parts of black paper oil, and 5˜-90 parts of base oil. An additional 10˜90 parts of cinnamon leaf oil, clary sage oil, clove bud oil, patchouli oil, petitgrain oil, bergamot oil or rose-otto oil, or their combination can be selectively added to the liquid composition.

### BACKGROUND OF THE INVENTION

(a) Field of the Invention

The present invention relates to essential oil technology and more particularly, to an aphrodisiac essential oil composition practical for directly applying to the body, or for use in a condom or paper towel to achieve the effect of increasing sexual desire. The invention relates also to an application method of using the aphrodisiac essential oil composition in condoms and tissue paper.

(b) Description of the Prior Art

Aphrodisiac products currently available on the market are mainly made in the form of an oral drug. However, taking an aphrodisiac oral solution can cause side effects, such as headache, facial flushing, indigestion, muscle pain, blurred vision, nasal congestion and dizziness, etc. Patients with cardiovascular, liver or kidney dysfunction patients should not take aphrodisiac oral products. When compared with aphrodisiac oral products, using a smear technique to achieve the effect of helping increasing sexual desire is a more secure way. Many literatures expressed specific plant oils have aphrodisiac effects.

Peppermint oil is one of important therapeutic essential oils. Peppermint has a strong, clean, fresh, minty aroma. It is a highly regarded herb for soothing digestion and providing supportive effects on the liver and respiratory systems. Peppermint can also improve taste and smell when inhaled. It has a good antibacterial effect on pulmonary tuberculosis. Peppermint is also very good for treating dry cough and a variety of stones. It also has the ability of improving concentration and mental sharpness and the ability of directly affecting the brain's satiety centers.

Ginger is a flowering plant indigenous to southern China, widely used as a spice or a medicine. Ginger oil is commonly used for treating motion sickness, morning sickness, colic, upset stomach, gas, diarrhea, nausea, vomiting, loss of appetite, as well as pain relief from arthritis or muscle soreness, menstrual pain, upper respiratory tract infections, cough, and bronchitis. Ginger oil is also sometimes used for reducing chest pain, low back pain, stomach pain, and pain in some people with a form of arthritis. Further, ginger oil also has the ability of warming up the body and helping increasing sexual desire.

Tea tree is a plant native to Southeast Queensland and the Northeast coast of New South Wales, Australia. Tee tree oil is an extract of tea tree oil used traditionally as a topical antiseptic and antifungal treatment. It can be used for controlling the symptoms of acne. Tee tree oil appears to be effective with toenail fungus and possibly athlete's foot. Tee tree oil is also used for other conditions such as gum disease, vaginal infections, and dandruff. Tea tree oil is known for its topical antiseptic and anti-fungal treatment or infection-reducing benefits. It also has antibiotic properties and helps aid against aliments such as skin infections, rashes, burns and much more.

Black paper is produced from the still-green unripe drupes of the pepper plant. The drupes are cooked briefly in hot water, both to clean them and to prepare them for drying. It is native to south India, and is extensively cultivated there and elsewhere in tropical regions. Black paper oil essential oil is a warm and spicy essential oil that helps to increase warmth of the body and mind, relieving sore muscles and joints, boost the immune and digestive system, stimulate the kidneys and disperse bruising by increasing circulation to the skin. The aroma is energizing and stimulating, helping increasing sexual desire.

Cinnamon leaf is a natural aphrodisiac. Cinnamon leaf oil has antiseptic, analgesic, antispasmodic, antibiotic, astringent, carminative, aphrodisiac, cardiac and other benefits. It can be used to treat general pain, respiratory tract infections, rheumatism and arthritis. Feelings of exhaustion and depression can be eased by using this essential oil.

Clary sage oil is an essential oil extracted by steam distillation from the buds and leaves of the clary sage oil plant and often used to support the hormonal system. Clary sage oil is commonly mixed with juniper, lavender and sandalwood oil for application. In the manufacture of perfume, clary sage oil is often used as spice fixative. Clary sage oil can be attributed to its properties as an antidepressant, anticonvulsive, antispasmodic, antiseptic, aphrodisiac, astringent, bactericidal, carminative, deodorant, digestive, emenagogue, euphoric, hypotensive, nervine, sedative, stomachic and uterine substance.

Clove bud oil is best known for its association with dental practices. Clove bud oil can be used to counteract mouth and throat irritation and pain when applied directly to the skin. Clove bud oil can also be used in aromatherapy to aid with digestive problems. Clove bud oil also helps alleviate muscular aches and sprains, and also helps relieve muscle aches and pains. Clove bud oil offers a powerful action against gas and bloating. It reduces gas pressure in the stomach, aiding in the proper elimination of food and toxins. Clove bud oil also relieves the discomfort of peptic ulcers and is effective against nausea, hiccups, motion sickness and vomiting. Adding 3-5 drops of clove bud oil in the vaporizer can produce a good bactericidal effect. Using clove bud oil in winter will make the body warm and more resistant against bacteria.

Patchouli oil has a strong effect of boosting libido and can promote wound healing. Patchouli oil has been used in perfumes and also in incense, skin care, insect repellents, and alternative medicines. Patchouli oil has also been used as an aphrodisiac, and also helps reduce tension, insomnia and anxiety, while uplifting the mind. Although patchouli oil is not so hot and so burning like mustard and pepper, but it gives the impression of positive oil. Patchouli oil works as a stimulant at a small dosage, and has a calming effect when a high dosage. The positive effect of patchouli oil is most obvious in symptoms of nervousness. It is a very strong nerve stimulant to keep you awake all night like when you drink a lot of liquid ginseng.

Petitgrain oil is an essential oil steam distilled from the same botanical species as neroli and bitter orange, and widely used in perfumery and aromatherapy as fresh-scented essential oils. Traditionally, petitgrain oil has been used to support a healthy nervous system and to improve instances of excessive perspiration and oily skin and hair.

Bergamot essential oil can act on the gastrointestinal tract to stop spasms, eliminate flatulence and help digestion, and therefore it is suitable for treating abdominal cramps, bloating and indigestion. Bergamot essential oil appears to be effective with eczema, psoriasis, acne, slow healing wounds and ulcer. It promotes wound healing. Bergamot essential oil can also be used for treating seborrheic dermatitis and dandruff caused by seborrheic dermatitis. Further, bergamot essential oil is also curative for scabies. Bergamot essential oil can be attributed to its properties as a deodorant. Further, bergamot essential oil has a calming effect, can relieve human anxiety. The fresh scent of bergamot essential oil can make people comfortable. Further, bergamot essential oil is a fragrance for the famous earl grey tea.

Rose-otto essential oil is an intoxicating aroma. The origin of the cultivated rose is often quoted as the Gulf of Persia. In early days, the rose industry was developed and dominated by Persia. Rose-otto essential oil is effective for strengthening body resistance to consolidate constitution. It can be used to treat body fluid loss. Oral rose-otto oil can help male increase sperm and promote the regeneration of body cells, i.e., rose is a known libido recovery agent, and therefore, rose-otto oil is suitable for treating impotence or infertility, and this indication has been included in Indian pharmacopoeias. Rose has a profound influence on women's sexual function, i.e., the function of organ adjustment, making the sexual organ to restore its original function.

### SUMMARY OF THE INVENTION

The present invention has been accomplished under the circumstances in view. It is therefore the main object of the present invention to provide an aphrodisiac essential oil composition, which can be sprayed by a female user to specific part of the body to help increase sexual desire, eliminating the side effect problem of the conventional method of taking an oral medication.

To achieve this and other objects of the present invention, an aphrodisiac essential oil composition is prepared by mixing 10˜90 parts of peppermint oil, 10˜90 parts of ginger oil, 10˜90 parts of tea tree oil, 10˜90 parts of black paper oil and 5˜90 parts of base oil into a liquid mixture for applying to the body directly.

Preferably, the aphrodisiac essential oil composition can also be further mixed with a substance selected from the group cinnamon leaf oil, clary sage oil, clove bud oil, patchouli oil, petitgrain oil, bergamot oil, rose-otto oil, and their combinations.

The invention further provides an aphrodisiac essential oil composition application method by mixing a predetermined amount of the aphrodisiac essential oil composition prepared as described above with a condom and then packing the aphrodisiac essential oil composition-contained condom in a pack so that when the user open the condom for application, the condom has the aphrodisiac essential oil composition contained therein to help increase sexual desire.

The invention further provides another aphrodisiac essential oil composition application method by using a paper towel to absorb the aphrodisiac essential oil composition prepared as described above, and then rubbing the paper towel over the body to accurately control the applied amount the aphrodisiac essential oil composition, preventing excessive use of the aphrodisiac essential oil composition and achieving the effect of increasing sexual desire.

### DETAILED DESCRIPTION OF THE PREFERRED EMBODIMENTS

An aphrodisiac essential oil composition in accordance with the present invention is prepared by mixing peppermint oil, ginger oil, tea tree oil, black paper oil, and base oil that is selected from the group of grape seed oil, sweet almond oil and silicone oil. Exemplars of the preparation of the aphrodisiac essential oil composition are outlined hereinafter:

### EXAMPLE 1

Take 10˜90 parts of peppermint oil, 10 parts of ginger oil, 10 parts of tea tree oil, 10 parts of black paper oil and 5 parts of base oil, and then well mix these substances into a liquid mixture.

### EXAMPLE 2

Take 10˜90 parts of peppermint oil, 20˜90 parts of ginger oil, 10 parts of tea tree oil, 10 parts of black paper oil and 5 parts of base oil, and then well mix these substances into a liquid mixture.

### EXAMPLE 3

Take 10˜90 parts of peppermint oil, 10˜90 parts of ginger oil, 20˜90 parts of tea tree oil, 10 parts of black paper oil and 5 parts of base oil, and then well mix these substances into a liquid mixture.

### EXAMPLE 4

Take 10˜90 parts of peppermint oil, 10˜90 parts of ginger oil, 10˜90 parts of tea tree oil, 20˜90 parts of black paper oil and 5 parts of base oil, and then well mix these substances into a liquid mixture.

### EXAMPLE 5

Take 10˜90 parts of peppermint oil, 10˜90 parts of ginger oil, 10˜90 parts of tea tree oil, 10˜90 parts of black paper oil and 10˜90 parts of base oil, and then well mix these substances into a liquid mixture.

Further, cinnamon leaf oil, clary sage oil, clove bud oil, patchouli oil, petitgrain oil, bergamot oil, rose-otto oil, and their combinations can be selectively added to the aphrodisiac essential oil composition that is prepared by peppermint oil, ginger oil, tea tree oil, black paper oil and base oil.

Exemplars of this preparation are explained as follows:

### EXAMPLE 6

Take 10˜90 parts of peppermint oil, 10˜90 parts of ginger oil, 10˜90 parts of tea tree oil, 10˜90 parts of black paper oil, 5 parts of base oil and 10˜90 parts of cinnamon leaf oil, and then well mix these substances into a liquid mixture.

### EXAMPLE 7

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of clary sage oil, and then well mix these substances into a liquid mixture.

### EXAMPLE 8

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of clove bud oil, and then well mix these substances into a liquid mixture.

### EXAMPLE 9

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of patchouli oil, and then well mix these substances into a liquid mixture.

### EXAMPLE 10

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of petitgrain oil, and then well mix these substances into a liquid mixture.

### EXAMPLE 11

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of bergamot, and then well mix these substances into a liquid mixture.

### EXAMPLE 12

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil and 10˜90 parts of rose-otto oil, and then well mix these substances into a liquid mixture.

### EXAMPLE 13

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil and 10˜90 parts of clove bud oil, and then well mix these substances into a liquid mixture.

### EXAMPLE 14

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil and 10˜90 parts of patchouli oil, and then well mix these substances into a liquid mixture.

### EXAMPLE 15

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil and 10˜90 parts of petitgrain oil, and then well mix these substances into a liquid mixture.

### EXAMPLE 16

Take 10˜90 parts of peppermint oil, 10˜90 parts of ginger oil, 10˜90 parts of tea tree oil, 10˜90 parts of black paper oil, 5˜90parts of base oil, 10˜90 parts of cinnamon leaf oil, 10˜90 parts of clary sage oil and 10˜90 parts of bergamot, and then well mix these substances into a liquid mixture.

### EXAMPLE 17

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil and 10˜90 parts of rose-otto oil, and then well mix these substances into a liquid mixture.

### EXAMPLE 18

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil and 10˜90 parts of patchouli oil, and then well mix these substances into a liquid mixture.

### EXAMPLE 19

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil and 10˜90 parts of petitgrain oil, and then well mix these substances into a liquid mixture.

### EXAMPLE 20

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil and 10˜90 parts of bergamot oil, and then well mix these substances into a liquid mixture.

### EXAMPLE 21

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil and 10˜90 parts of rose-otto oil, and then well mix these substances into a liquid mixture.

### EXAMPLE 22

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil and 10˜90 parts of petitgrain oil, and then well mix these substances into a liquid mixture.

### EXAMPLE 23

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil, 10˜90 parts of patchouli oil and 10˜90 parts of bergamot oil, and then well mix these substances into a liquid mixture.

### EXAMPLE 24

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil, 10˜90 parts of patchouli oil and 10˜90 parts of rose-otto oil, and then well mix these substances into a liquid mixture.

### EXAMPLE 25

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil, 10˜90 parts of patchouli oil, 10˜90 parts of petitgrain oil and 10˜90 parts of bergamot oil, and then well mix these substances into a liquid mixture.

### EXAMPLE 26

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil, 10˜90 parts of patchouli oil, 10˜90 parts of petitgrain oil and 10˜90 parts of rose-otto oil, and then well mix these substances into a liquid mixture.

### EXAMPLE 27

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil, 10˜90 parts of clove bud oil, 10˜90 parts of patchouli oil, 10˜90 parts of petitgrain oil, 10˜90 parts of bergamot oil and 10˜90 parts of rose-otto oil, and then well mix these substances into a liquid mixture.

### EXAMPLE 28

Take 10˜90 parts of peppermint oil, 10˜90 parts of ginger oil, 10˜90 parts of tea tree oil, 10˜90 parts of black paper oil, 5˜90 parts of base oil and 10˜90 parts of clary sage oil, and then well mix these substances into a liquid mixture.

### EXAMPLE 29

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil and 10˜90 parts of clove bud oil, and then well mix these substances into a liquid mixture.

### EXAMPLE 30

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil and 10˜90 parts of patchouli oil, and then well mix these substances into a liquid mixture.

### EXAMPLE 31

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil and 10˜90 parts of petitgrain oil, and then well mix these substances into a liquid mixture.

### EXAMPLE 32

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil and 10˜90 parts of bergamot oil, and then well mix these substances into a liquid mixture.

### EXAMPLE 33

Take 10˜90 parts of peppermint oil, 10˜90 parts of ginger oil, 10˜90 parts of tea tree oil, 10˜90 parts of black paper oil, 5˜90 parts of base oil, 10˜90 parts of cinnamon leaf oil, 10˜90 parts of clary sage oil and 10˜90 parts of rose-otto oil, and then well mix these substances into a liquid mixture.

### EXAMPLE 34

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil and 10˜90 parts of patchouli oil, and then well mix these substances into a liquid mixture.

### EXAMPLE 35

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil and 10˜90 parts of petitgrain oil, and then well mix these substances into a liquid mixture.

### EXAMPLE 36

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil and 10˜90 parts of bergamot oil, and then well mix these substances into a liquid mixture.

### EXAMPLE 37

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil and 10˜90 parts of rose-otto oil, and then well mix these substances into a liquid mixture.

### EXAMPLE 38

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil and 10˜90 parts of petitgrain oil, and then well mix these substances into a liquid mixture.

### EXAMPLE 39

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil and 10˜90 parts of bergamot oil, and then well mix these substances into a liquid mixture.

### EXAMPLE 40

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil and 10˜90 parts of rose-otto oil, and then well mix these substances into a liquid mixture.

### EXAMPLE 41

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil, 10˜90 parts of petitgrain oil and 10˜90 parts of bergamot oil, and then well mix these substances into a liquid mixture.

### EXAMPLE 42

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil, 10˜90 parts of petitgrain oil and 10˜90 parts of rose-otto oil, and then well mix these substances into a liquid mixture.

### EXAMPLE 43

Take 10˜90 parts of peppermint oil, 10˜90 parts of ginger oil, 10˜90 parts of tea tree oil, 10˜90 parts of black paper oil, 5˜90 parts of base oil, 10˜90 parts of clary sage oil, 10˜90 parts of clove bud oil, 10˜90 parts of patchouli oil, 10˜90 parts of petitgrain oil, 10˜90 parts of bergamot oil and 10˜90 parts of rose-otto oil, and then well mix these substances into a liquid mixture.

### EXAMPLE 44

Take 10˜90 parts of peppermint oil, 10˜90 parts of ginger oil, 10˜90 parts of tea tree oil, 10˜90 parts of black paper oil, 5˜90 parts of base oil and 10˜90 parts of clove bud oil, and then well mix these substances into a liquid mixture.

### EXAMPLE 45

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil and 10˜90 parts of patchouli oil, and then well mix these substances into a liquid mixture.

### EXAMPLE 46

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil and 10˜90 parts of petitgrain oil, and then well mix these substances into a liquid mixture.

### EXAMPLE 47

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil and 10˜90 parts of bergamot oil, and then well mix these substances into a liquid mixture.

### EXAMPLE 48

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil and 10˜90 parts of rose-otto oil, and then well mix these substances into a liquid mixture.

### EXAMPLE 49

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil and 10˜90 parts of petitgrain oil, and then well mix these substances into a liquid mixture.

### EXAMPLE 50

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil and 10˜90 parts of bergamot oil, and then well mix these substances into a liquid mixture.

### EXAMPLE 51

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil and 10˜90 parts of rose-otto oil, and then well mix these substances into a liquid mixture.

### EXAMPLE 52

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil, 10˜90 parts of petitgrain oil and 10˜90 parts of bergamot oil, and then well mix these substances into a liquid mixture.

### EXAMPLE 53

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil, 10˜90 parts of petitgrain oil and 10˜90 parts of rose-otto oil, and then well mix these substances into a liquid mixture.

### EXAMPLE 54

Take 10˜90 parts of peppermint oil, 10˜90 parts of ginger oil, 10˜90 parts of tea tree oil, 10˜90 parts of black paper oil, 5˜90 parts of base oil, 10˜90 parts of clove bud oil, 10˜90 parts of patchouli oil, 10˜90 parts of petitgrain oil, 10˜90 parts of bergamot oil and 10˜90 parts of rose-otto oil, and then well mix these substances into a liquid mixture.

### EXAMPLE 55

Take 10˜90 parts of peppermint oil, 10˜90 parts of ginger oil, 10˜90 parts of tea tree oil, 10˜90 parts of black paper oil, 5˜90 parts of base oil and 10˜90 parts of patchouli oil, and then well mix these substances into a liquid mixture.

### EXAMPLE 56

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil and 10˜90 parts of petitgrain oil, and then well mix these substances into a liquid mixture.

### EXAMPLE 57

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil and 10˜90 parts of bergamot oil, and then well mix these substances into a liquid mixture.

### EXAMPLE 58

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil and 10˜90 parts of rose-otto oil, and then well mix these substances into a liquid mixture.

### EXAMPLE 59

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil, 10˜90 parts of petitgrain oil and 10˜90 parts of bergamot oil, and then well mix these substances into a liquid mixture.

### EXAMPLE 60

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil, 10˜90 parts of petitgrain oil and 10˜90 parts of rose-otto oil, and then well mix these substances into a liquid mixture.

### EXAMPLE 61

Take 10˜90 parts of peppermint oil, 10˜90 parts of ginger oil, 10˜90 parts of tea tree oil, 10˜90 parts of black paper oil, 5˜90 parts of base oil, 10˜90 parts of patchouli oil, 10˜90 parts of petitgrain oil, 10˜90 parts of bergamot oil and 10˜90 parts of rose-otto oil, and then well mix these substances into a liquid mixture.

### EXAMPLE 62

Take 10˜90 parts of peppermint oil, 10˜90 parts of ginger oil, 10˜90 parts of tea tree oil, 10˜90 parts of black paper oil, 5˜90 parts of base oil and 10˜90 parts of petitgrain oil, and then well mix these substances into a liquid mixture.

### EXAMPLE 63

Take 10˜90 parts of peppermint oil, 10˜90 parts of ginger oil, 10˜90 parts of tea tree oil, 10˜90 parts of black paper oil, 5˜90 parts of base oil, 10˜90 parts of petitgrain oil and 10˜90 parts of bergamot oil, and then well mix these substances into a liquid mixture.

### EXAMPLE 64

Take 10˜90 parts of peppermint oil, 10˜90 parts of ginger oil, 10˜90 parts of tea tree oil, 10˜90 parts of black paper oil, 5˜90 parts of base oil, 10˜90 parts of petitgrain oil and 10˜90 parts of rose-otto oil, and then well mix these substances into a liquid mixture.

### EXAMPLE 65

Take 10˜90 parts of peppermint oil, 10˜90 parts of ginger oil, 10˜90 parts of tea tree oil, 10˜90 parts of black paper oil, 5˜90 parts of base oil, 10˜90 parts of petitgrain oil, 10˜90 parts of bergamot oil and 10˜90 parts of rose-otto oil, and then well mix these substances into a liquid mixture.

### EXAMPLE 66

Take 10˜90 parts of peppermint oil, 10˜90 parts of ginger oil, 10˜90 parts of tea tree oil, 10˜90 parts of black paper oil, 5˜90 parts of base oil and 10˜90 parts of bergamot oil, and then well mix these substances into a liquid mixture.

### EXAMPLE 67

Take 10˜90 parts of peppermint oil, 10˜90 parts of ginger oil, 10˜90 parts of tea tree oil, 10˜90 parts of black paper oil, 5˜90 parts of base oil, 10˜90 parts of bergamot oil and 10˜90 parts of rose-otto oil, and then well mix these substances into a liquid mixture.

### EXAMPLE 68

Take 10˜90 parts of peppermint oil, 10˜90 parts of ginger oil, 10˜90 parts of tea tree oil, 10˜90 parts of black paper oil, 5˜90 parts of base oil and 10˜90 parts of rose-otto oil, and then well mix these substances into a liquid mixture.

After preparation, the aphrodisiac essential oil composition is filled in a bottle for allowing consumers to purchase and to use. When in use, drip a few drops of the aphrodisiac essential oil composition onto the hand, and then smear the aphrodisiac essential oil composition over the back of the ear, the neck, the armpits, the breasts, the thighs or the skin area around the genital organ of the female, and then message the applied area with the fingers for 2 to 3 minutes, achieving an effect of helping increasing sexual desire.

Further, the invention also provides an aphrodisiac essential oil composition application method. An example of this aphrodisiac essential oil composition application method is to take a predetermined amount of the aforesaid aphrodisiac essential oil composition, and then to mix the taken amount of the aforesaid aphrodisiac essential oil composition with a condom, and then to pack the aphrodisiac essential oil composition-contained condom in a pack for application. For condom application, the base oil of the aphrodisiac essential oil composition is restricted to silicone oil. Grape seed oil and sweet almond oil are not suitable for used as base oil the aphrodisiac essential oil composition. Thus, when the user opens the condom and attaches the condom to the genital organ, the aphrodisiac essential oil composition is directly applied to the genital organ without further application procedures, achieving the effects of application amount control and ease of use.

The invention also provides an alternate form of the aphrodisiac essential oil composition application method. This alternate form is to mix a predetermined amount of the prepared aphrodisiac essential oil composition with at least one piece of paper towel, and then to pack the at least one piece of paper towel in a pack. The paper towel can be non-woven fabric or tissue paper. Thus, when the user open the paper towel pack, the user can pick up one piece of the paper towel having the aphrodisiac essential oil composition absorbed therein, and then rub the piece of paper towel over the back of the ear, the neck, the armpits, the breasts, the thighs or the skin area around the genital organ, and then message the applied area with the fingers for 2 to 3 minutes, achieving an effect of helping increasing sexual desire. Using a paper towel to absorb the prepared aphrodisiac essential oil composition for application facilitates the use of the aphrodisiac essential oil composition and the control of the applied amount of the aphrodisiac essential oil composition.

1. An aphrodisiac essential oil composition, comprising: 10˜90 parts of peppermint oil, 10˜90 parts of ginger oil, 10˜90 parts of tea tree oil, 10˜90 parts of black paper oil, and 5˜90 parts of base oil, said base oil being silicone oil.

2. The aphrodisiac essential oil composition as claimed in claim 1, further comprising 10˜90 parts of cinnamon leaf oil.

3. The aphrodisiac essential oil composition as claimed in claim 1, further comprising 10˜90 parts of clary sage oil.

4. The aphrodisiac essential oil composition as claimed in claim 1, further comprising 10˜90 parts of clove bud oil.

5. The aphrodisiac essential oil composition as claimed in claim 1, further comprising 10˜90 parts of patchouli oil.

6. The aphrodisiac essential oil composition as claimed in claim 1, further comprising 10˜90 parts of petitgrain oil.

7. The aphrodisiac essential oil composition as claimed in claim 1, further comprising 10˜90 parts of bergamot oil.

8. The aphrodisiac essential oil composition as claimed in claim 1, further comprising 10˜90 parts of rose-otto oil.

9. An aphrodisiac essential oil composition application method, comprising the steps of:
mixing 10˜90 parts of peppermint oil, 10˜90 parts of ginger oil, 10˜90 parts of tea tree oil and 10˜90 parts of black paper oil with 5˜90 parts of base oil of silicone oil to form an aphrodisiac essential oil composition; and
packing a predetermined amount of said aphrodisiac essential oil composition with a condom in a pack.

10. An aphrodisiac essential oil composition application method, comprising the steps of:
mixing 10˜90 parts of peppermint oil, 10˜90 parts of ginger oil, 10˜90 parts of tea tree oil and 10˜90 parts of black paper oil with 5˜90 parts of base oil to form an aphrodisiac essential oil composition, wherein said base oil is selected from the group consisting of grape seed oil, sweet almond oil and silicone oil; and
packing a predetermined amount of said aphrodisiac essential oil composition with a paper towel in a pack.

11. The aphrodisiac essential oil composition application method as claimed in claim 10, wherein said paper towel is selected from the group consisting of non-woven fabric and tissue paper.
